(19) United States
(12) Patent Application Publication
(10) Pub. No.: US 2002/0182717 A1
(43) Pub. Date: Dec. 5, 2002

(54) Method of preparing supported lipid film membranes and use thereof

(75) Inventor: Olof Karlsson, Uppsala (SE)

Correspondence Address: SEED INTELLECTUAL PROPERTY LAW GROUP PLLC, 701 FIFTH AVE, SUITE 6300, SEATTLE, WA, 98104-7092, US

(73) Assignee: Biacore AB, Uppsala (SE)

(21) Appl. No.: 10/098,739

(22) Filed: Mar. 14, 2002

Related U.S. Application Data
(60) Provisional application No. 60/276,729, filed on Mar. 16, 2001.

(30) Foreign Application Priority Data
Mar. 14, 2001 (SE) 0100875-4

(51) Int. Cl. G01N 33/542; B05D 3/00; C12M 1/34; G01N 33/53

(52) U.S. Cl. 435/287.2; 427/2.11; 435/7.9

(57) ABSTRACT

The present invention relates to a method of preparing a substrate surface supporting a lipid film membrane structure, which method comprises the steps of contacting a substrate surface with an aqueous liquid containing detergent/lipid mixed micelles to adhere detergent/lipid mixed micelles to the substrate surface, and then contacting the substrate surface having detergent/lipid mixed micelles adhered thereto with an aqueous liquid substantially free from detergent to elute the detergent molecules from the adhered mixed micelles and make the remaining lipid molecules assemble into a lipid film membrane structure on the substrate surface. The invention also relates to a substrate supporting a lipid film membrane structure as prepared by the method, the use of a substrate supporting a lipid film membrane structure as prepared by the method for molecular interaction studies, and a substrate surface for use in the method.

### BACKGROUND OF THE INVENTION

1. Field of the Invention

The present invention relates generally to a preparation method for lipid film membrane type structures on substrate surfaces, particularly sensor surfaces, a supported lipid film membrane prepared by the method, the use of such supported lipid film membranes in lipid membrane interaction studies, and a substrate surface for use in the method.

2. Description of the Related Art

Membranes play a central role in the structure and function of all living cells. Defining the boundaries of the cells as well as compartments within the cells, the membranes not only have a barrier function but also control the transport of substances across the membrane, mediate information between the compartments and are the site of enzymatic reactions.

The membranes consist of a lipid molecule bilayer with proteins and other components, such as e.g., lipopolysaccharides. The membrane lipids have a polar (hydrophilic) part and a non-polar (hydrophobic) part. In the membrane bilayer, the non-polar parts of the lipids are turned towards each other in the middle of the bilayer with the polar lipid parts forming the external surfaces of the membrane. Proteins in the membranes, which may extend through the membrane (transmembrane proteins) or be anchored to one of the membrane surfaces, are generally bound to the membrane through noncovalent forces, such as the hydrophobic force or electrostatic interactions, but there are also examples of proteins that are covalently bound to lipids.

Artificial supported bilayer lipid membranes (so-called BLM's) on solids provide a natural environment for the immobilization of proteins, and have therefore gained increasing interest for application on biosensors of various kinds. Such BLM's may be used for studying, for example, ligand-receptor interactions at the membrane-water interface.

In order to obtain suitable conditions for transmembrane proteins, it is desirable to provide for an aqueous layer between the membrane and the support. For example, EP-A-441120 proposes to join the lower lipid layer to the support by hydrophilic spacer arms.

The assembly of BLM's on a solid support may be obtained in various ways. Basically, however, three different approaches have generally been used.

In a first approach, a solid support is immersed into an aqueous solution and a lipid monolayer is then formed at the air-water interface. When the support is retracted from the solution, a lipid monolayer is adhered to the support. The support is then re-immersed into the solution which results in the formation of a lipid bilayer on the support (Suarez-Isla, B. A., et al., Biochemistry (1983) 22:2319-2323).

In a second approach, a solid support surface is contacted with an aqueous solution of lipid vesicles whereby lipid bilayers under certain conditions may be formed by spontaneous fusion of the lipid vesicles, or liposomes, to the surface (a liposome is a lipid bilayer enclosing a volume). In this way, for example, fluid lipid bilayers floating on a thin water film may be formed on a hydrophilic support, such as SiO₂. Similarly, lipid bilayer membranes may be produced which rest on an ultrathin hydrated polymer film, or hydrogel (see e.g., Sackmann, E., Science (1996) 271:43-48). On the other hand, liposomes contacted with a hydrophobic substrate will build up a monolayer on the hydrophobic surface (Cooper, M. A., et al., Biochim. Biophys. Acta (1998) 1373:101-111).

The membrane may be linked more stably to the support, for instance, through hydrophilic spacers as mentioned above, or as disclosed in, for example, U.S. Pat. No. 5,922,594 by covalently binding the lipid bilayer to a self-assembled monolayer of straight long chain molecules coated on the substrate surface. In the latter case, an aqueous solution of micellar or vesicle liposomes is contacted with the monolayer-supporting surface so that a majority of the liposomes bind covalently to the monolayer to form an anchored bilayer lipid membrane. Due to the covalent anchoring of the membrane to the support, the process may be performed in a flow cell using a controlled laminar flow. In a variant of preparing bound lipid bilayers, a streptavidin-coated surface is contacted with biotinylated liposomes.

In a third approach, a solid substrate surface is contacted with an aqueous solution of mixed micelles formed by codispersion of detergent with lipid in a receptacle, and the formation of a lipid bilayer on the surface is then effected by selective removal of the detergent by either diluting the micellar solution with buffer, “the micellar dilution technique”, or by dialysis.

Micellar dilution may be exemplified by Lang, H., et al., Langmuir (1994) 10:197-210, where a supported mixed bilayer was formed by contacting a gold electrode supporting a thiolipid monolayer with an aqueous solution of mixed micelles of lipid and detergent, and then stepwise diluting the solutions with electrolyte below the critical micelle concentration (CMC) of the detergent.

Removal of the detergent by dialysis is disclosed in, for example, U.S. Pat. No. 5,204,239, where mixed phospholipid/detergent micelles in solution were allowed to attach to a gold electrode surface via a bridging arm with a terminal thiol group, and the detergent was then removed by dialysis, which resulted in the formation of a continuous lipid bilayer attached to the electrode.

Supported lipid bilayer membranes containing proteins or peptides (oligo- or polypeptides) have been prepared by including the protein or peptide in the lipid layer and lipid vesicles used in the first and second approaches, respectively, mentioned above, or in the mixed micelles used in the above third approach.

Heyse, S., et al., Biochem. (1998) 37:507-522, describe functionalization of a gold surface with a patterned organic monolayer consisting of alternating regions with carboxyl-exposing thiols (CTA) and regions with hydrocarbon-exposing thiolipids. Rhodopsin-containing mixed phospholipid/detergent micelles were applied to the patterned monolayer, and on dilution with buffer, the phospholipids were self-assembled on the structured support and formed membranes that alternated between phospholipid bilayer domains (on CTA) and monolayer domains (on thiolipids), rhodopsin preferentially being in the bilayer domains.

Bieri, C., et al., Nature Biotechnology (1999) 17:1105-1108, describe covering a sensor chip by a self-assembled monolayer consisting of biotinylated thiols and an excess of ω-hydroxy-undecanethiol to which streptavidin was bound. Biotinylated protein along with lipid/detergent mixed micelles were added to the surface, whereupon a supported lipid bilayer was formed by micellar dilution as above, the biotinylated protein binding to the immobilized streptavidin.

WO 96/38726 discloses a solid device having a covalently attached coating of a lipid bilayer containing a protein. A proximal phospholipid layer, which may contain e.g., a transmembrane protein, was first covalently attached to a linker layer on the solid device. A distal lipid layer was then deposited by vesicle or mixed micelle fusion to give a lipid bilayer structure. Mixed micelle fusion was effected by depositing a mixed micelle dispersion onto the proximal phospholipid layer, and then diluting with an aqueous buffer.

U.S. Pat. No. 5,765,355 discloses preparation of a bilayer lipid membrane sensor by contacting a gold surface with a thiolipid/detergent solution to covalently bind a thiolipid layer to the surface. After washing with detergent solution, a transmembrane protein-containing phospholipid/detergent solution was added and stepwise diluted with potassium chloride solution. The resulting lipid bilayer consisted of a mixed monolayer of thiolipid and phospholipid, and a second phospholipid monolayer containing the transmembrane protein.

Generally, however, the prior art methods for preparing lipid films, and especially bilayers, on solid surfaces are laborious, time-consuming and do not always give the desired result. Also, as regards receptor-containing cell membrane preparations, the receptor density in such preparations has usually not been high enough for successful use in biosensor applications.

There is therefore a need of improved methods for preparing lipid film membranes on solids, and especially for preparing such membranes containing reconstituted proteins or peptides.

### BRIEF SUMMARY OF THE INVENTION

It is an object of the present invention to provide a method of preparing supported lipid film membrane structures, which method overcomes the disadvantages of the prior art methods and which thus is easy and rapid to carry out and which makes it possible to provide substrate surfaces densely packed with reconstituted membrane proteins.

According to the present invention, it has now been found that the above object as well as other objects and advantages may be achieved by a method wherein a detergent/lipid mixed micelle preparation is deposited from an aqueous dispersion thereof onto a substrate surface, and the substrate surface is then contacted with an aqueous liquid substantially free from detergent to elute detergent from the micelles, the remaining lipid molecules thereby forming a lipid film on the substrate surface.

Therefore, in one aspect, the present invention provides a method of preparing a substrate surface supporting a lipid film membrane structure, which method comprises the steps of:

a) contacting a substrate surface with an aqueous liquid containing detergent/lipid mixed micelles to adhere detergent/lipid mixed micelles to the substrate surface, and then

b) contacting the substrate surface having detergent/lipid mixed micelles adhered thereto with an aqueous liquid substantially free from detergent to elute the detergent molecules from the adhered mixed micelles and make the remaining lipid molecules assemble into a lipid film membrane structure on the substrate surface.

In another aspect, the invention provides a substrate supporting a lipid film membrane structure as prepared according to the first aspect above.

In yet another aspect, the invention provides the use of the method for reconstituting membrane protein function.

In still another aspect, the invention provides the use of a substrate supporting a lipid film membrane structure as prepared according to the first aspect above for interaction studies with membrane associated proteins or peptides, e.g., in screening of drug candidate molecules.

In yet another aspect, the invention provides a substrate surface for use in the method.

These and other aspects of the invention will be evident upon reference to the following detailed description and the attached drawings.

### BRIEF DESCRIPTION OF THE SEVERAL VIEWS OF THE DRAWING(S)

FIGS. 1A to 1C are a schematic illustrations of the reconstitution of membrane proteins by deposition of detergent/lipid mixed micelles on an amphiphilic biosensor sensing surface with immobilized proteins, and subsequent elution of the detergent to form a lipid bilayer.

FIG. 2 is a sensorgram showing the response (RU) versus time (s) for the injection of three different mixtures of lipid and detergent into the flow cell of a SPR-biosensor instrument.

FIG. 3 is a diagram wherein the ratio of the difference between the concentration of detergent (OG) and its CMC to the concentration of lipid (POPC) is plotted against the deposition level of lipid on a sensing surface when passed by aqueous samples of detergent/lipid mixed micelles.

FIG. 4 is a diagram representing the signaling of reconstituted rhodopsin receptor on a SPR biosensor sensing surface. The relative response (RU) is plotted against time (s) for the illumination of a POPC-reconstituted rhodopsin surface (1) relative to the signal in a POPC-only reference surface (2).

### DETAILED DESCRIPTION OF THE INVENTION

As mentioned above, the present invention relates to the preparation f lipid film membranes, especially lipid bilayer membranes, supported on a solid substrate surface. Membrane lipids are amphiphilic molecules, or amphiphiles, comprising a hydrophilic (water soluble) part and a hydrophobic (water insoluble) part. The lipids form together a characteristic bilayer where the hydrophobic parts are directed towards the middle and the hydrophilic parts form the two surfaces of the membrane. In this bilayer, there may also be biomolecules, such as proteins or peptides, partly or fully inserted therein.

The invention is based on the idea of reconstituting lipid membranes, with or without proteins or peptides, by adhering mixed micelles of detergent and lipid to a substrate surface with subsequent depletion of the detergent by aqueous liquid substantially free from detergent. When detergent/lipid mixed micelles in an aqueous solution are contacted with the substrate surface, they attach thereto. With proper detergent/lipid ratio, this attachment is strong enough to allow selective elution of the detergent from the adhered detergent/lipid mixed micelles even when the liquid containing mixed micelles (and detergent) is rapidly replaced by aqueous liquid substantially free from detergent, such as by a liquid flow.

The replacement of the mixed micelle-containing liquid by detergent-free liquid may be performed in a static system by removal of the micelle-containing liquid from the substrate surface, e.g., by a pipette, with subsequent addition of the detergent-free liquid to the substrate surface. Alternatively, the substrate having detergent/lipid mixed micelles adhered thereto is moved to another compartment or receptacle with detergent-free liquid. Preferably, however, the depletion of the detergent from the substrate surface is performed by a liquid flow over the substrate surface.

If proteins or peptides are attached to the substrate surface prior to forming the lipid film thereon, the proteins or peptides are reconstituted with lipids when the detergent is selectively eluted from the adhered detergent/lipid mixed micelles.

The type of lipid film membrane that is formed on the substrate surface, i.e., a monolayer or bilayer, depends mainly on the character of the surface. While the method of the invention performed on a hydrophobic substrate surface usually will produce a lipid monolayer, the formation of a lipid bilayer, which is (at least currently) preferred, requires an amphiphilic substrate surface as will be explained in more detail below.

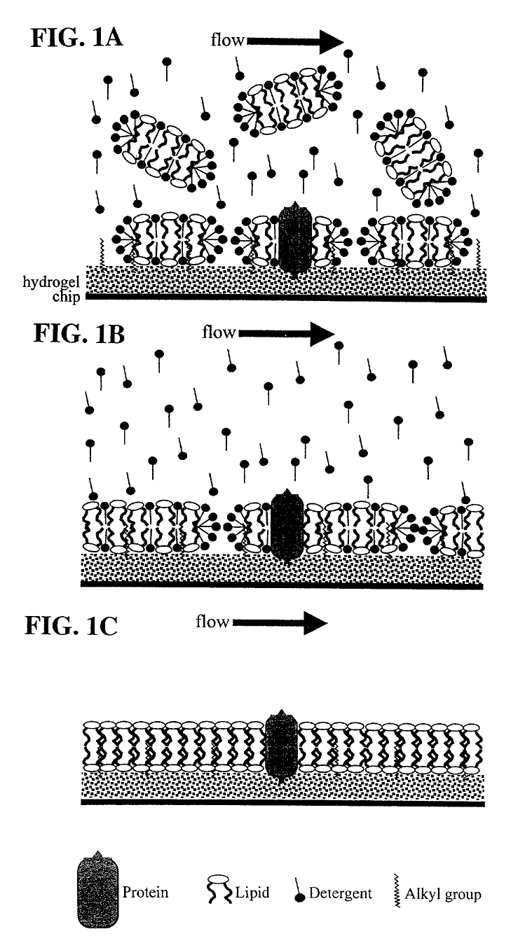

The procedure of the invention applied to the preparation of a supported lipid bilayer membrane is schematically illustrated in FIGS. 1A to 1C. Mixed micelles consisting of detergent and lipid are attached to projecting (hydrophobic) alkyl groups and other non-polar groups like hydrophobic parts of immobilized membrane proteins (for example, a pre-immobilized receptor protein) on an otherwise hydrophilic sensor surface (FIG. 1A). When a liquid flow free from amphiphile (detergent) is injected over the sensor surface, a detergent monomer concentration is maintained in the mobile phase leading to a rapid depletion of detergent from the mixed micelles (FIG. 1B). The lipids remain attached to the surface, assembling into a bilayer structure, and are able to reconstitute the function of membrane proteins on the sensor surface (FIG. 1C).

The term substrate, or support, as used herein refers to any material body or layer onto which it is desired to apply a lipid film membrane. Exemplary substrate materials are gels, beads, polymers, stationary hydrophobic or amphiphilic phases, etc. Reference to the “surface” of the substrate or support includes, for porous substrates, the interior surfaces as well. Currently preferred substrates are sensor surfaces and chromatographic particles.

As mentioned above, the substrate surface should be amphiphilic for a lipid bilayer to be formed. The term “amphiphilic” is, however, to be construed broadly herein. Basically, the term means that the surface should exhibit hydrophilic and hydrophobic chemical structures (i.e., chemical groups or residues, including whole molecules, e.g., biomolecules) in ratios that may vary within a wide range, including surfaces ranging from partially hydrophobic to partially hydrophilic.

Preferably, the hydrophobic structures of an amphiphilic surface constitute chemical projections capable of interacting with hydrophobic parts of the lipid bilayer. For example, the surface may support a self-assembled layer of hydrophilic residues mixed with hydrophobic residues (e.g.,alkyl groups), the latter preferably extending out from the hydrophilic residues. As mentioned above, the hydrophobic residues serve to adhere the mixed micelles whereas the hydrophilic residues aid in the formation of the lipid bilayer upon the depletion of the detergent.

As mentioned above and will be further described below, it is often desired that the supported membrane bilayer includes a biomolecule, such as a membrane protein or peptide (the term peptide including oligopeptides and polypeptides). Such proteins or peptides may be the species that provide the amphiphilic character to an otherwise hydrophilic surface. For example, a hydrophobic protein or peptide may be attached to a self-assembled layer of hydrophilic residues supported on the substrate surface. Alternatively, the amphiphilic character of the surface may be provided by both hydrophobic chemical groups and membrane proteins or peptides.

It is believed that the meanings of the terms “hydrophilic” and “hydrophobic” are well known to those skilled in the art. Basically, hydrophobic may be defined as water-repelling whereas hydrophilic may be defined as water-attracting. It is also customary to define hydrophilicity and hydrophobicity with regard to the contact angle for a droplet of a liquid on a planar solid surface, the contact angle being measured from the plane of the surface, tangent to the water surface at the three phase boundary line. A hydrophilic liquid will thus have a low contact angle on a hydrophilic surface, whereas a hydrophobic liquid will have a high contact angle. For example, hydrophobic surfaces typically have contact angles in the range of 40 to 110°, while the contact angles with water for hydrophilic surfaces typically are in the range of 1 to 25°.

The optimum ratio of hydrophilic moieties to hydrophobic moieties on the amphiphilic substrate surface will depend on the particular moieties as well as on the components of the mixed micelles used and may readily be determined by the skilled person for each particular situation.

A presently preferred substrate surface comprises a biocompatible porous matrix, preferably a hydrogel, modified to contain a certain amount of hydrophobic groups. For the present purposes, a “hydrogel” may be defined as presenting a surface layer of bound molecules which by reason of their chemical nature hold a large fraction of water, in which the molecules are predominantly in an amorphous, water-solvated state, and in which the thickness of the layer is of the order of 30 Å minimum up to any indefinitely higher limit (Merrill, E. W., et al., Hydrogels in Medicine and Pharmacy, Vol. III, N. A. Peppas, Ed., Chapter I, CRC Press, Inc., Boca Raton, Fla. (1986)). Building reconstituted membranes on polymer hydrogels will allow essentially undisturbed lipid bilayer dynamics. An exemplary such modified hydrogel is a carboxymethyl-modified dextran polymer hydrogel on which a substantial fraction of the glucose moieties have been modified by alkyl groups.

Before describing the additional features of the invention in more detail, the terms micelle, mixed micelle, lipid, and detergent will be discussed.

“Micelles” are aggregates of amphiphilic molecules (amphiphiles), such as detergents, which aggregates do not enclose an aqueous volume. They are formed when the concentration of the amphiphilic molecule in a liquid exceeds a critical value called the “critical micelle concentration”, or CMC. By strict definition, the CMC is the concentration when 50% of the amphiphiles are in the form of micelles, but CMC is often used as the concentration where micelles first appear. The micelles are usually globular but also other shapes, such as e.g., rod-shaped micelles, may form at high amphiphile concentrations.

“Detergents” are in a broad sense defined as substances capable of lowering the surface tension of liquids, but the term relates in a more narrow sense to surfactants as means for purification purposes. As mentioned above, the detergents are amphiphiles and may form micelles. They may be anionic, cationic, zwitterionic or uncharged. In the context of the present invention, detergents may be said to be micelle-forming amphiphiles.

“Lipids” are generally esters of long-chain carboxylic esters and include fats, waxes and cell lipids. In the context of the present invention, the lipids are usually membrane lipids (cell and organelle membrane lipids), which exhibit an enormous diversity, are more or less amphiphilic and include inter alia the following classes: phospholipids, lysophospholipids, glycosyl diacylglycerols, plasmalogens, sphingomyelins, gangliosides, and sterols. Depending on the chemical structure of the cell lipids and physical factors like temperature, pH and ionic strength, they may form different types of aggregates in aqueous media. For example, lysophospholipids form micelles, whereas some phospholipids under certain conditions form bilayers.

If a detergent (or detergent mixture) is co-dispersed with a membrane lipid (or lipid membrane mixture), “mixed micelles” may be formed consisting of alternating bilayer-prone lipid molecules and detergent molecules. As mentioned above, a lipid bilayer will form on an amphiphilic support if the detergent is eluted from the micelles, such as by a continuous flow of substantially detergent-free liquid.

Lipids suitable for reconstitution purposes according to the present invention may readily be selected by the skilled person and are basically those able to (i) form lamellar aggregation structures, at least somewhere in the temperature interval 15-40° C.; and (ii) be solubilised by any suitable detergent to constitute a nonturbid micellar suspension. These lipids, or lipid mixtures, should preferably have a relatively low CMC and may be selected from natural or synthetic lipids or mixtures thereof.

Generally, such lipids may be selected from natural or synthetic lipid molecules such as glycerophospholipids, glyceroglycolipids, sphingophospholipids and sphingoglycolipids, and from the classes phosphatidyl choline, phosphatidyl ethanolamine, phosphatidyl serine, phosphatidyl glycerol, phosphatidyl acid, phosphatidyl inositol, galactopyranoside, glucopyranoside, digalactopyranoside, diglucopyranoside, ceramide-phosphatidyl choline, ceramide-phosphatidyl ethanolamine, ceramide-phosphatidyl serine, ceramide-phosphatidyl glycerol, ceramide-phosphatidyl acid, ceramide-phosphatidyl inositol, sphingomyelin molecules, glucosylceramides, glucocerebrosides, galactoceramides, galactocerebrosides, gangliosides, monoacyl phosphatidyl choline, cardiolipin molecules, that may be linked to saturated or mono-, di or polyunsaturated fatty or fluorocarbon chains ranging from three to thirty carbons in length where fatty chains attached to the head group can be the same or of different structure, cholesterol, lanosterol, ergosterol, stigmasterol, sitosterol and derivatives thereof capable of being incorporated into lipid membranes, N,N-dimethyl-N-octadecyl-1-octadecanammonium chloride or bromide, (N-[1-(2,3-dioleoyloxy)propyl]-N,N,N-trimethylammonium chloride, N-[2,3-dihexadecyloxy)prop-1-yl]-N,N,N-trimethylammonium chloride, bolaamphiphiles, polyglycerolmonoalkylethers, polyethoxymonoalkylethers, and liposome-forming molecules from the classes amphiphilic polymers, amino acids, crown ether compounds and di(acyloxy)dialkylsilanes, as well as mixtures of the lipids mentioned above.

Specific exemples of such lipids are: Phosphatidylcholines with acyl chains ranging in length from 14 to 18 carbons, such as di-1,2-myristoyl-SN-phosphatidylcholine, di-1,2-oleoyl-SN-phosphatidylcholine, 1-palmitoyl-2-oleoyl-SN-phosphatidylcholine (POPC) and 1-stearoyl-2-oleoyl-SN-phosphatidylcholine; glyceroglycolipids like di-1,2-myristoyl-3-diglucopyranosyl-SN-glycerol, di-1,2-oleoyl-3-diglucopyranosyl-SN-glycerol, 1-palmitoyl-2-oleoyl-3-diglucopyranosyl-SN-glycerol, 1-stearoyl-2-oleoyl-3-diglucopyranosyl-SN-glycerol, di-1,2-myristoyl-3-digalactopyranosyl-SN-glycerol, di-1,2-oleoyl-3-digalactopyranosyl-SN-glycerol, 1-palmitoyl-2-oleoyl-3-digalactopyranosyl-SN-glycerol and 1-stearoyl-2-oleoyl-3-digalactopyranosyl-SN-glycerol, or sphingomyelins with the corresponding acyl chain lengths and unsaturations as those mentioned above.

Likewise, detergents, or detergent mixtures, suitable for use in the present invention may readily be selected by the skilled person. Generally, it is preferred that the CMC of the detergent is relatively high. Thus, while the detergent CMC usually should be at least about 1 mM, it is often preferable that the CMC is higher, such as at least about 10 mM.

Exemplary detergents are 3-[(3-cholamidopropyl)dimethylammoniol]-1-propane sulfonate (CHAPS), 3-[(3-cholamidopropyl)dimethylammonio]-2-hydroxy-1-propane sulfonate (CHAPSO), N,N-bis-(3-D-gluconeamidopropyl)-deoxycholamide (deoxy-BIGCHAP), sodium taurocholate, cholic acid, deoxycholic acid, n-octylglucoside (OG), n-octylthioglucoside, N-decyl-N,N-dimethyl-3-ammonio-1-propane sulfonate (Zwittergent 3-10), N-dodecyl-N,N-dimethyl-3-ammonio-1-propane sulfonate (Zwittergent 3-12), octanoyl-N-methylglucamide (MEGA-8), decanoyl-N-methylglucamide (MEGA-10), 6-O-(N-heptylcarbamoyl)-methyl-α-D-glucopyranoside (HECAMEG), and sucrose monolaurate.

The selective elution of detergent from the mixed micelles in accordance with step c) in the method of the invention as defined above is basically driven by the difference in critical micelle concentration, or CMC, of the detergent and lipid, respectively. Thus, generally, the higher this difference is, the better. This is due to the relatively high CMC of the detergent striving to maintain a high monomer concentration in the mobile phase and thereby getting extracted from the stationary phase.

The ratio of detergent to lipid in the mixed micelles may be a critical factor in the method of the invention. Too detergent rich preparations may result in poor or no attachment at all of mixed micelles to the surface (note that liquids of a high detergent concentration, well above the CMC, are usually used to completely wash away deposited lipids from a sensor surface in a flow cell). On the other hand, too lipid rich preparations tend to be turbid and adhere slowly to the solid support, building aberrant aggregate structures. Therefore, it is crucial that the lipid/detergent ratio in the mixed micelles be balanced to obtain a maximal level of attachment to the solid support while still having a clear, non-turbid solution.

In the prior art time-consuming micellar dilution methods, a functional detergent/lipid ratio will be reached at some point during the dilution, providing for the formation of a lipid bilayer (although too large dilution steps will give vesicle formation). In contrast, the approach of the present invention is to use mixed micelles of a predetermined detergent/lipid ratio known to give a clear micellar solution which attaches the micelles to the surface sufficiently strongly not to be released when contacted with detergent-free solution.

The desired ratio may be determined by relating the ratio of the excess concentration of detergent over CMC, i.e., ([detergent]—CMC)/[lipid], to the amount of lipid deposited on the solid support. The optimum ratio varies depending on the particular lipid and detergent selected but can readily be determined by the skilled person, as will be further described below. Usually, the above ratio is in the range of from about 0.1 to about 100, preferably from about 0.5 to about 100, and more preferably from about 0.5 to about 10. In preferred embodiments, the optimum ratio may be in the range from about 0.5 to about 5, an exemplary range being from about 0.5 to about 3, which is applicable to inter alia octylglucoside and POPC.

The concentration of lipid (or lipids) is usually in the range of from about 0.1 to about 50 mM, preferably from about 0.1 to about 10 mM.

The concentration of detergent (or detergents) is usually in the range of from about 0.5×CMC to about 10×CMC, preferably from about 0.5×CMC to about 5×CMC for the detergent.

The method of the invention should generally be performed at conditions, such as e.g., temperature, where the lipid (or lipid mixture) may form a liquid lamellar phase, i.e., above the main transition temperature of the lipid (or lipid mixture). In many cases it may be satisfactory to carry out the method at room temperature.

The term “elute” is used herein basically in the commonly acknowledged sense, i e., to denote the removal of an adsorbed substance from an adsorbent by means of a solvent, the adsorbent in the present case being the substrate surface.

The liquid used for eluting the detergent molecules from the mixed micelles should be substantially free from detergent, which means that the eluent liquid should contain no detergent at all or contain only trace amounts thereof. Otherwise the composition of the eluent liquid may vary depending on the particular application of the method of the invention. For example, in the case of chromatographic applications, the eluent liquid may be a conventional type eluent, and when the method is used in flow-cell-based biosensor applications, the eluent liquid may be the conventionally used running buffer.

The eluent liquid should provide a mobile phase for detergent removal, a salient feature of the invention being that micelle-containing liquid above the substrate surface is completely replaced (e.g., displaced) rather than diluted by the detergent-free liquid. The flow of eluent liquid should be substantially continuous, i.e., it should preferably not be arrested, and if so, at least not for any long time periods, and it should also not be interrupted by air bubbles. Likewise, while the liquid flow rate may vary during the elution, it is preferred that the flow rate is substantially constant. The optimum flow rate, which may vary within wide limits, depends on inter alia the flow system used and may readily be chosen by the skilled person for each particular situation.

While it is possible to carry out the deposition of the mixed micelles on the substrate surface according to step a) of the method in a stationary liquid, and only perform the elution step b) under flow conditions, it is preferred that the whole procedure is performed using a continuous liquid flow.

As mentioned above, the method of the invention may be used for chromatographic applications. To this end, the lipid bilayer membranes may be applied to hydrophobic or amphiphilic chromatographic particles, preferably in place in a column or in a channel of a micro- or nanofluidic device. In the latter types of devices, the lipid bilayer membranes may alternatively be applied to a channel wall.

Currently, however, it is preferred that the method of the invention is applied to a biosensor. As appreciated by those skilled in the art, a biosensor is an analytical device for analyzing minute quantities of sample solution having an analyte of interest, wherein the interaction of the analyte with a sensing surface is detected by a detection device. For the purposes of the invention, the sensing surface or surfaces of the biosensor are preferably located in a flow cell or flow cells, i.e., broadly a channel part(s) or compartment(s) through which a liquid flow may be maintained.

For many chromatographic as well as biosensor applications, it is desired that the lipid bilayer contains a biomolecule(s), usually a protein or peptide, preferably a so-called membrane protein. As mentioned above, a protein or peptide may be applied to the bilayer lipid membrane by including the protein or peptide in the mixed micelle preparation to be deposited on the substrate surface, or by attaching the protein or peptide to the bilayer lipid membrane after the formation thereof, e.g., by adsorption or covalent binding. For many applications, however, it may be preferable to attach the protein or peptide to the substrate surface before depositing the mixed micelle preparation thereon. The lipid bilayer will then reconstitute the protein or peptide by being formed around the protein or peptide molecules. In this way, a high protein or peptide density may be ensured. As mentioned above, the protein or peptide may then constitute the sole hydrophobic elements of an amphiphilic surface.

Immobilization of the biomolecule to the substrate surface may be performed by methods well known in the art. For instance, groups on a protein or peptide may be coupled directly to active functional groups on the substrate, for example by amine coupling to surface carboxyl groups as described in the Examples below. It may, however, at least in some cases be advantageous to attach the biomolecule, such as a protein or peptide, to the substrate surface via a coupling member, such as, for instance, a spacer, a linker, or another protein or peptide. Such spacers, linkers etc are well-known to the skilled person and need not be discussed in any detail herein. More particularly, a specific binding pair (sbp) may be used for attaching the biomolecule to the substrate surface. Exemplary specific binding pairs include antibody-antigen, antibody-hapten, biotin-avidin (or streptavidin), carbohydrate-protein, carbohydrate-lectin, nucleic acid duplexes, oligonucleotide pairs, oligonucleotide-polynucleotide pairs, polynucleotide pairs, such as DNA-DNA and DNA-RNA, protein nucleic acid (PNA) pairs, protein-RNA, interacting peptide pairs, and protein-metal chelate.

For example, G-protein coupled receptors (GPCRs) can be selectively biotinylated in their N-terminal glycosylations (Bieri, C., et al., Nature Biotech. (1999) 17:1105-1108) and subsequently captured on the substrate surface by immobilized streptavidin, which could be followed by reconstitution of the receptors on the surface according to the invention, so-called on surface reconstitution. This would give a uniform orientation of the lipid-reconstituted receptors, facilitating studies of their interactions with intracellular components. Likewise, a histidine-tagged protein or peptide (e.g., a recombinant fusion protein) may be captured on the substrate surface by an immobilized metal chelate, such as, e.g., a nitrilotriacetic acid (NTA) nickel complex.

“Antibody” as used herein means an immunoglobulin which may be natural or partly or wholly synthetically produced and also includes active fragments, including Fab antigen-binding fragments, univalent fragments and bivalent fragments. The term also covers any protein having a binding domain that is homologous to an immunoglobulin binding domain. Such proteins can be derived from natural sources, or partly or wholly synthetically produced. Exemplary antibodies are the immunoglobulin isotypes and the Fab, Fab′, F(ab′)₂, scFv, Fv, dAb, and Fd fragments.

“Hapten” as used herein means a low molecular species that may give rise to an immune response only when coupled to a larger molecule or cell or by aggregation. After immunisation, however, free haptens may react with antibodies.

A substrate surface having an array of different (or the same) proteins or peptides in a lipid bilayer membrane may be prepared by attaching these proteins or peptides to the substrate surface, and then depositing a mixed micelle preparation to the surface to reconstitute the proteins or peptides.

“On surface reconstitution” according to the present invention permits the same handling of membrane proteins as for soluble proteins by standard procedures. No reconstitutions, into more or less stable proteoliposomes, have to be prepared in advance and when the protein is ready for reconstitution on a substrate surface, no elaborate detergent dilution steps are needed.

Should it, for some reason, be desired that the lipid bilayer membrane contain other biomolecules than proteins or peptides, such molecules may be provided in the same ways as those outlined for proteins and peptides.

Several analytical applications of lipid bilayer membranes use a lipid bilayer without any protein or peptide. For example, the published U.S. patent application Ser. No. 2002/0019019 A1 (the full disclosure of which is incorporated by reference herein) discloses a method of assaying drug candidates (usually small molecules) with regard to inter alia absorption, i.e., the uptake of a drug compound from the site of administration into the systemic circulation, by estimating the absorption from biosensor data associated with a sensor chip having lipids immobilized thereon. Specifically, the analysis of interactions of a drug candidate with immobilized liposomes can be used to predict whether or not the drug candidate is absorbed by the small intestine. The method of the invention may advantageously be used for such absorption assaying of drug candidates.

A great advantage of the present method of invention is the promptness by which the lipid reconstitution can be accomplished. Thus, whilst the immobilization of liposomes to a sensor surface is rather time-consuming, the present process of forming a lipid bilayer membrane on a support surface may be performed in a very short time. This means among other things that a fresh lipid bilayer may be formed for each assay, which may be desired for several reasons. For example, an accidentally introduced air bubble may tear away a part of the bilayer, it may not be possible to completely wash away absorbed small molecules (candidate molecules often tend to remain bound to the lipid bilayer in ligand binding assays), etc. Many approaches that before were either very laborious or impossible have now become both possible and easy to carry out. This is important for applications demanding standardized and high throughput processing, like screening and array assays.

As mentioned above, the method of the present invention is, at least currently, considered to be especially useful for chromatographic and biosensor applications, in particular biosensor applications. Biosensors may be based on a variety of detection methods. Typically such methods include, but are not limited to, mass detection methods, such as piezoelectric, optical, thermo-optical and surface acoustic wave (SAW) device methods, and electrochemical methods, such as potentiometric, conductometric, amperometric and capacitance methods. With regard to optical detection methods, representative methods include those that detect mass surface concentration, such as reflection-optical methods, including both internal and external reflection methods, angle, wavelength or phase resolved, for example ellipsometry and evanescent wave spectroscopy (EWS), the latter including surface plasmon resonance (SPR) spectroscopy, Brewster angle refractometry, critical angle refractometry, frustrated total reflection (FTR), evanescent wave ellipsometry, scattered total internal reflection (STIR), optical wave guide sensors, evanescent wave-based imaging such as critical angle resolved imaging, Brewster angle resolved imaging, SPR angle resolved imaging, and the like. Further, photometric methods based on, for example, evanescent fluorescence (TIRF) and phosphorescence may also be employed, as well as waveguide interferometers.

In the detailed description and Examples that follow, the present invention is illustrated, by way of example only, in the context of surface plasmon resonance (SPR) spectroscopy. One exemplary type of SPR-based biosensors is sold by Biacore AB (Uppsala, Sweden) under the trade name BIACORE® (hereinafter referred to as “the BIACORE instrument”). These biosensors utilize a SPR based mass-sensing technique to provide a “real-time” binding interaction analysis between a surface bound ligand and an analyte of interest.

The BIACORE instrument includes a light emitting diode (LED), a sensor chip including a glass plate covered with a thin gold film, an integrated fluid cartridge providing a liquid flow over the sensor chip, and a photo detector. Incoming light from the LED is totally internally reflected at the glass/gold interface and detected by the photo detector. At a certain angle of incidence (“the SPR angle”), a surface plasmon wave is set up in the gold layer, which is detected as an intensity loss “or dip” in the reflected light. More particularly, and as is appreciated by those skilled in the art, the phenomenon of SPR associated with the BIACORE instrument is dependent on the resonant coupling of monochromatic p-polarized light, incident on a thin metal film via a prism and a glass plate, to oscillations of the conducting electrons, called plasmons, at the metal film on the other side of the glass plate. These oscillations give rise to an evanescent field that extends a distance of the order of one wavelength (≈1 μm) from the surface into the liquid flow. When resonance occurs, light energy is lost to the metal film through a collective excitation of electrons therein and the reflected light intensity drops at a sharply defined angle of incidence, the SPR angle, which is dependent on the refractive index within reach of the evanescent field in the proximity of the metal surface.

As noted above, the SPR angle depends on the refractive index of the medium close to the gold layer. In the BIACORE instrument, dextran is typically coupled to the gold surface, with the analyte-binding ligand being bound to the surface of the dextran layer. The analyte of interest is injected in solution form onto the sensor surface through the fluid cartridge. Because the refractive index in the proximity of the gold films depends on (i) the refractive index of the solution (which is constant), and (ii) the amount of material bound to the surface, the binding interaction between the bound ligand and analyte can be monitored as a function of the change in SPR angle. In the Examples below embodying the present invention, a lipid bilayer membrane is bound to a modified such dextran layer.

A typical output from the BIACORE instrument is a “sensorgram”, which is a plot of response (measured in “resonance units” or “RU”) as a function of time. An increase of 1,000 RU corresponds to an increase of mass on the sensor surface of about 1 ng/mm².

A detailed discussion of the technical aspects of the BIACORE instrument and the phenomenon of SPR may be found in U.S. Pat. No. 5,313,264. More detailed information on matrix coatings for biosensor sensing surfaces is given in, for example, U.S. Pat. Nos. 5,242,828 and 5,436,161. In addition, a detailed discussion of the technical aspects of the biosensor chips used in connection with the BIACORE instrument may be found in U.S. Pat. No. 5,492,840. The full disclosures of the above-mentioned U.S. patents are incorporated by reference herein.

In the following Examples, various aspects of the present invention are disclosed more specifically for purposes of illustration and not limitation.

### EXAMPLE 1

This example describes the preparation of mixed micelles of detergent and lipid, deposition of the mixed micelles on a sensor chip surface, and elution of detergent to form a lipid bilayer on the surface. A BIACORE 3000 instrument (Biacore AB, Uppsala, Sweden) was used. BIACORE instruments are based on surface plasmon resonance (SPR) detection at gold surfaces, and a micro-fluidic system is used for passing samples and running buffer through four individually detected flow cells (one by one or in series), with very high precision and with small sample volumes needed. As sensor chip was used Pioneer Chip L1 (Biacore AB, Uppsala, Sweden) which has a gold surface with a covalently linked carboxymethyl-modified dextran polymer hydrogel on which a substantial fraction of the glucose moieties is modified with alkyl groups (Cooper, M. A., et al., Anal. Biochem. (2000) 277:196-205). Running buffer was HBS-N (10 mM HEPES pH 7.4 and 150 mM NaCl) (Biacore AB, Uppsala, Sweden).

A. Preparation of Mixed Micelles

1-Palmitoyl-2-oleoyl-phosphatidylcholine (POPC) (lipid; Avanti Polar Lipids Inc., Alabaster, Ala., U.S.A.) at 10 mM in chloroform was pipetted to round bottomed glass tubes, pre-washed in chloroform. The solvent was evaporated under a stream of nitrogen gas and solvent remains were removed under reduced pressure for at least 2 h. Octylglucoside (OG) (detergent; Sigma, St. Louis, U.S.A.) was diluted from a 0.5 M stock (frozen in aliquots) to desired concentrations with HBS-N and water, yielding 9 mM HEPES pH 7.4 and 135 mM NaCl (HBS-OG). HBS-OG was added to the dry lipid film and the mixtures were shaken every 10 min for at least 45 min at room temperature. In this way a number of combinations with 0.12-10 mM POPC and 5-50 mM octylglucoside were prepared. The preparations were checked for turbidity by the eye.

B. On Surface Reconstitution of Lipid Membranes

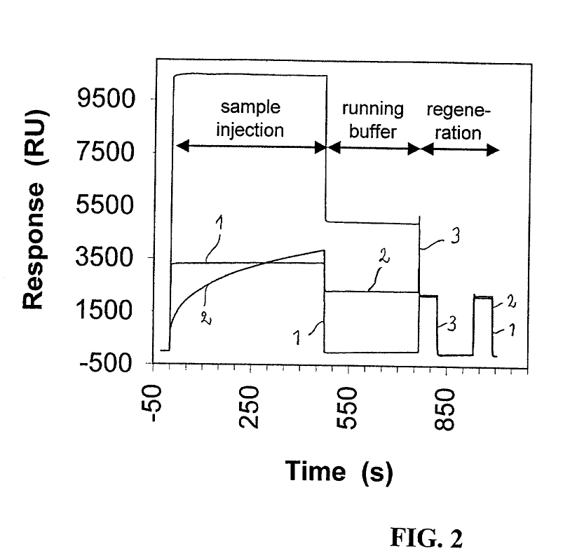

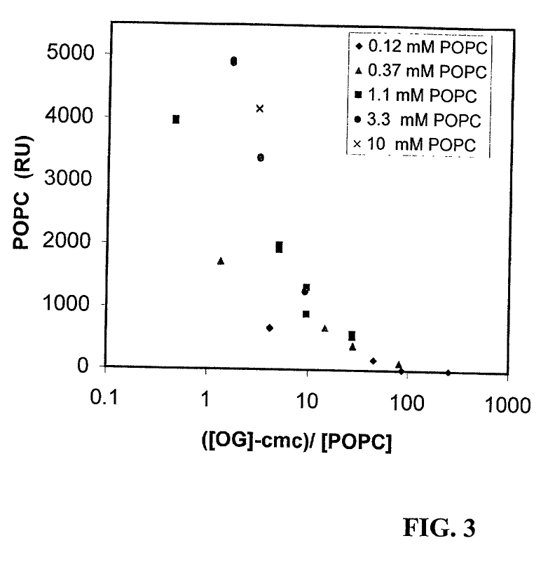

After conditioning of the L1-surface with five 30 s injections of 20 mM 3-[(3-cholamidopropyl)dimethylammonio]propanesulphonic acid (CHAPS) (Sigma, U.S.A.) the mixed micelles (prepared as described in step A above) were injected into BIACORE 3000 for 8 min (during optimization, otherwise 1 min) at 5 μl/min. The washing of the flow system was delayed for 2 min after injection. Lipid deposition quantity data were collected 100 s after the end of injection. The sensor surface was regenerated by two 1 min injections of 20 mM CHAPS or optimally 50 mM octylglucoside. The results are shown in FIGS. 2 and 3.

As seen in FIG. 2, three groups of samples could be identified: (1) Detergent rich clear preparations giving no attachment of POPC at all; (2) lipid rich, turbid samples that adhered firmly with slow association curves of analogous appearance as liposome capturing curves (Cooper, M. A., et al., supra; and Erb E. -M., et al., Anal. Biochem. (2000) 280:29-35); and (3) balanced, clear preparations leading to fast elution of octylglucoside and high deposition of POPC and having very steep association and dissociation curves, but with a significant and lasting increment of the baseline after the injection. This remaining signal elevation is most likely caused by deposited POPC on the sensor surface. The mechanism behind this can be explained in the following way: Mixed micelles of proper compositions adhere very rapidly to the amphiphilic surface during the injection. When the injection is terminated and detergent free buffer is running through the flow cell, the mixed micelles remain adhered to the amphiphilic hydrogel but the octylglucoside, striving to keep the monomer concentration (CMC), is very rapidly depleted. As the POPC micelles become detergent free, they fuse to build a continuous lipid film. This kind of reconstitution is taking advantage of the efficient fluidics of the instrument. Injections of 0.1 mg/ml of BSA showed that these lipid bilayers equally well protected the Pioneer Chip L1 surface from binding of BSA, as surfaces saturated with LUVs (large unilamellar vesicles). This together with the fact that the optimum deposition level of about 5000 RU is close to the theoretical level for a bilayer, 4600 RU (Cooper, M. A., et al, supra; Cooper, M. A., et al, Biochim. Biophys. Acta (1998) 1373:101-111; and Sévin-Landais, A., et al., Biophys. Chem. (2000) 85:141-152), indicates that the result is a continuous bilayer covering the entire sensor surface.

The ratio of detergent to lipid in the mixed micelles seems to be a major factor in determining to which of the three groups above the sample belongs. When the ([octylglucoside]-CMC)/[POPC] ratio is related to the amount of POPC deposited after injection, it appears that the optimum is between 0.5 and 3 octylglucoside molecules per POPC in the mixed micelles, as illustrated in FIG. 3 (only clear preparations are included in the diagram). Since this equation is dependent on the CMC of the detergent and the solubility of the current lipid mixture, which can vary considerably (Schürholz, T., Biophys. Chem. (1996) 58:87-96), the fine-tuning of composition has to be done in each instance. In this case, the best mixture found was 3.3 mM POPC and 25 mM octylglucoside. It also seems to be of high importance that the detergent is rapidly depleted after the injection. When different concentrations of octylglucoside were included in the running buffer, no deposition at all occurred or the system became very unstable.

When using lipids other than POPC, by themselves or in mixture with POPC, shifts in the optimal ([octylglucoside]-CMC)/[POPC] ratio were detected. However, most of the variants could mediate as high lipid deposition levels as could pure POPC, as shown in the following Table 1.

TABLE 1

| | Optimal detergent/lipid | Lipid deposition level |
|---|---|---|
| Lipid composition | ratio* | Relative POPC (%) |
| POPC | 7.3 | 100 |
| DOPC | 7.5 | 95 |
| DOPC/POPC (50:50) | 7.3 | 124 |
| POPE/POPC (25:75) | 8.0 | 100 |
| POPG/POPC (25:75) | 6.7 | 93 |
| POPS/POPC (25:75) | 7.3 | 80 |

*The CMC is not subtracted from the octylglucoside concentration since it varies with the lipid composition.

### EXAMPLE 2

This example describes immobilization of rhodopsin (a G-protein coupled receptor; GPCR) on a sensor surface, and reconstitution of the protein by the formation of a lipid bilayer on the surface. Rhodopsin function was tested by assaying its signaling capacity (transducin activation). BIACORE 3000 and BIACORE X instruments (Biacore AB, Uppsala, Sweden) were used. As sensor chip was used Pioneer Chip L1 (see Example 1 above).

A. Immobilization of Rhodopsin

After docking in BIACORE 3000 or BIACORE X (Biacore AB, Uppsala, Sweden), sensor chip Pioneer Chip L1 (Biacore AB, Uppsala, Sweden) was first washed by two 1 min injections of 20 mM CHAPS (Sigma, U.S.A.). The carboxymethyl-modified dextran polymer, which is partially substituted with alkyl groups on Pioneer Chip L1, was activated with an injection of 0.2 M N-ethyl-N-dimethylamino-propylcarbodiimide (EDC) and 50 mM N-hydroxysuccinimide (NHS) for 7 min. The running buffer was as in Example 1 above. Rhodopsin (obtained from Dr. Andreas von Usedom, Institute for Medical Physics and Biophysics, Humboldt University, Berlin) stored at 61 μM in 20 mM BTP, 130 mM NaCl, 1 mM MgCl₂, 53 mM octylglucoside and 200 mM α-methyl-mannopyranoside, was diluted to 0.61 μM in 10 mM maleate pH 6.0 and 20 mM octylglucoside. The diluted rhodopsin was injected for 14 min minimum and the surface was then blocked by 0.96 M ethanolamine-HCl pH 8.5 and 20 mM octylglucoside (Sigma, St. Louis, MO, U.S.A.) for 7 min.

The amine coupling of rhodopsin above resulted in immobilization levels close to 4000 RU on the L1 chip, which corresponds to 4 ng/mm² (Stenberg, E., et al., J. Colloid Interface Sci. 143, 513-526) or 0.1 pmol/mm² of rhodopsin. Since the amine coupling is not site specific but can involve any free amine group on the protein (mostly lysine residues), the rhodopsin is not uniformly oriented on the chip surface. However, most of the lysines in rhodopsin are positioned on the C-terminal side (cytosolic side) according to sequence based models of the structure (Hargrave, P. A., et al., Biophys. Struct. Mech. (1983) 9:235-244). Hence, the C-terminal side is probably dominating as the side of attachment, keeping the outside out orientation in favour.

B. Reconstitution of Rhodopsin by Lipid Bilayer Formation

The immobilized rhodopsin obtained in step A above was immediately reconstituted by a 2 min injection of mixed micelles, 3.3 mM POPC and 25 mM octylglucoside in HBS-N, prepared as in Example 1 above. Around 4500 RU of POPC were deposited. In a reference flow cell with unmodified Pioneer Chip L1-surface, about 5000 RU of lipid was simultaneously deposited. The always lower amount of lipids that bound in the rhodopsin flow cell is probably due to the space occupied by the immobilized protein, which indicates that lipid and protein coexist in each other's proximity.

The lipids deposited this way could be completely removed by two consecutive 1 min injections of 50 mM octylglucoside, and then resupplemented with a new 1 min injection of 3.3 mM POPC in 25 mM octylglucoside. This was indicated by the very stable responses achieved during 10 cycles of removal and supplementation.

C. Assay for Rhodopsin Functionality

In order to judge if the rhodopsin has a native and functional conformation after immobilization and supplementation with lipids as described in steps A and B above, its signaling capacity was assayed. When rhodopsin is activated by light, it transmits its signal by activating transducin. Activated transducin dissociates from the membrane under consumption of GTP (Heyse, S., et al., Biochemistry (1998) 37:507-522).

Measurements of transducin dissociation by activated rhodopsin were performed in a BIACORE X instrument into which an optical fiber had been inserted for illumination of the flow cells. The instrument temperature was set to 20° C. All the operations were performed under safe red light. Equal volumes of 11.6 μM transducin (obtained from Dr. Andreas von Usedom, Institute for Medical Physics and Biophysics, Humboldt University, Berlin) and 1 mM GTP, both stored in 20 mM BTP, 130 mM NaCl and 1 mM MgCl₂, were mixed with three volumes of HBS-MDE (10 mM HEPES pH 7.4 and 150 mM NaCl, 1 mM MgCl₂, 1 mM DTT and 0.2 μM EDTA), giving a five times dilution of both (2.3 μm transducin and 200 mM GTP).

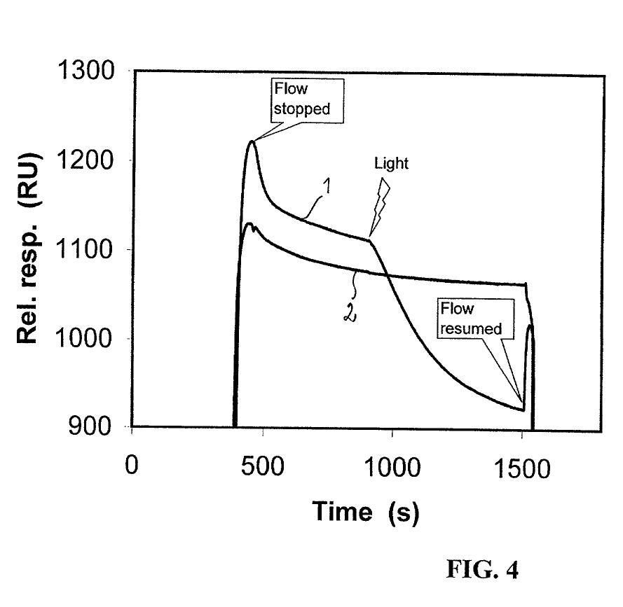

This solution was injected at 5 μl/min over both the rhodopsin and the reference flow cells in the manual mode. Upon injection, transducin bound readily to both the rhodopsin-POPC surface and the POPC reference surface, but when the injection was finished it dissociated very rapidly from the surface into the mobile phase of running buffer. In order to detect the active dissociation of transducin, the flow was stopped at maximal binding during the injection. FIG. 4 shows the response signal for the POPC-rhodopsin reconstituted surface (curve 1) relative to the signal for the POPC reference surface. The flow was stopped at 459 s (FIG. 4). A few minutes after the flow-stop, at 902 s (FIG. 4), the level of bound transducin was stabilized in both the reference and rhodopsin flow cells. The flow cells were then illuminated with an Ocean Optics 5 W halogen lamp via an optic fibre. The activation of the receptor was recorded as a surface mass decrease caused by dissociation of the activated transducin from the membrane. Since this decrease was not observed in the reference flow cell or in absence of GTP it was concluded that it displays the signalling capacity of the receptor. After the signal decrease had levelled out significantly, the flow was resumed and the injection terminated (FIG. 4). When recharging the rhodopsin on the sensor surface, 9-cis-retinal (Sigma, St. Louis, Mo., U.S.A.) at 10 μM in HBS-MDE and 0.7% DMSO were injected over both flow cells for 13 min. Membrane bound retinal was allowed to dissociate for about 40 min before next round of the assay.

Following extended illumination of the rhodopsin and over night incubation, the signaling capacity of the receptor was exhausted. However, after injection of 10 μM 9-cis-retinal a recovery of the signalling capacity was detected. This showed that also the ligand binding capacity of the receptor was preserved when reconstituted by the method presented here.

From these results it was concluded that rhodopsin in a micellar environment can be covalently attached to the Pioneer Chip L1 chip surface by a commonly employed protocol for protein immobilization without irreversible loss of function. Since rhodopsin is sensitive to its lipid environment (Brown, M. F., Chem. Phys. Lipids (1994) 73:159-180) and demands reconstitution with phospholipids (Bubis, J., Biol. Res. (1998) 31:59-71), it was concluded that the lipid deposition method described in Examples 1 and 2 can be generally used for functional reconstitution of membrane proteins on amphiphilic surfaces. The method can also be used generally for building lipid bilayers on amphiphilic surfaces in flow systems, and would also be applicable to chromatography columns.

From the foregoing it will be appreciated that, although specific embodiments of the invention have been described herein for purposes of illustration, various modifications may be made without deviating from the spirit and scope of the invention. Accordingly, the invention is not limited except as by the appended claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A method of preparing a substrate surface supporting a lipid film membrane structure, which method comprises the steps of:
a) contacting the substrate surface with an aqueous liquid containing detergent/lipid mixed micelles to adhere detergent/lipid mixed micelles to the substrate surface, and
b) contacting the substrate surface having detergent/lipid mixed micelles adhered thereto with an aqueous liquid substantially free from detergent to elute the detergent molecules from the adhered mixed micelles and make the remaining lipid molecules assemble into a lipid film membrane structure on the substrate surface.

2. The method according to claim 1, wherein in step b) the substrate surface is contacted with an aqueous liquid flow to elute the detergent molecules.

3. The method according to claim 2, wherein the liquid flow is substantially continuous.

4. The method according to claim 3, wherein the rate of said flow is substantially constant during the elution.

5. The method according to claim 2, wherein in step a) the substrate surface is contacted with an aqueous liquid flow containing detergent/lipid mixed micelles.

6. The method according to claim 5, wherein the liquid flow is substantially continuous.

7. The method according to claim 6, wherein the rate of the liquid flow is substantially constant.

8. The method according to claim 1, wherein the substrate surface is provided in a flow cell.

9. The method according to claim 8, wherein the substrate surface is part of the interior surface of the flow cell.

10. The method according to claim 1, wherein the substrate surface is provided in a chromatographic system.

11. The method according to claim 10, wherein the chromatographic system comprises chromatographic particles.

12. The method according to claim 1, wherein the substrate surface is a biosensor sensing surface.

13. The method according to claim 8, wherein the substrate surface is a biosensor sensing surface.

14. The method according to claim 1, wherein prior to step a) the substrate surface has a biomolecule immobilized thereon, which biomolecule is reconstituted in step b).

15. The method according to claim 14, wherein the biomolecule is selected from proteins and peptides.

16. The method according to claim 14, wherein different biomolecules are attached to respective different parts of the substrate surface, such that after step b) the substrate surface exhibits an array of reconstituted biomolecules.

17. The method according to claim 14, wherein the biomolecules are attached to the substrate surface via a coupling member.

18. The method according to claim 17, wherein the coupling member is selected from a spacer, a linker, a protein and a peptide.

19. The method according to claim 14, wherein each biomolecule is immobilized to the substrate surface via a specific binding pair, one member of the specific binding pair being attached to the substrate surface and the other member of the specific binding pair being part of or attached to the biomolecule.

20. The method according to claim 19, wherein the member of the specific binding pair that is attached to the substrate surface is an antibody directed to the biomolecule.

21. The method according to claim 19, wherein the member of the specific binding pair that is attached to the substrate surface is selected from avidin and streptavidin and the biomolecule is biotinylated.

22. The method according to claim 19, wherein the member of the specific binding pair that is attached to the substrate surface is a metal chelate and the biomolecule contains neighbouring histidine residues.

23. The method according to claim 1, wherein in step a) the substrate surface is contacted with a liquid containing a biomolecule which is reconstituted in step b).

24. The method according to claim 1, wherein the substrate surface comprises a hydrogel.

25. The method according to claim 24, wherein the hydrogel comprises a dextran polymer.

26. The method according to claim 1, wherein the substrate surface in step a) is amphiphilic.

27. The method according to claim 26, wherein the amphiphilic substrate surface comprises a hydrogel containing hydrophobic chemical groups.

28. The method according to claim 27, wherein the hydrogel comprises a carboxymethyl-modified dextran polymer hydrogel on which a fraction of the modified glucose moieties are substituted with alkyl groups.

29. The method according to claim 26, wherein the substrate surface comprises a hydrophobic biomolecule, and the amphiphilic substrate surface comprises an otherwise hydrophilic surface made amphiphilic through said biomolecule.

30. The method according to claim 29, wherein the biomolecule is selected from membrane proteins and peptides.

31. The method according to claim 26, wherein the amphiphilic substrate surface comprises a hydrophilic surface made amphiphilic by co-existing hydrophobic chemical groups and hydrophobic biomolecules.

32. The method according to claim 26, wherein a lipid bilayer membrane structure is formed on the substrate surface.

33. The method according to claim 1, wherein the ratio ([detergent]-CMC)/[lipid] is in the range from about 0.1 to about 100.

34. The method according to claim 1, wherein the ratio ([detergent]-CMC)/[lipid] is in the range from about 0.5 to about 100.

35. The method according to claim 1, wherein the ratio ([detergent]-CMC)/[lipid] is in the range from about 0.5 to about 10.

36. The method according to claim 1, wherein the ratio ([detergent]-CMC)/[lipid] is in the range from about 0.5 to about 5.

37. The method according to claim 1, wherein the lipid is selected from natural or synthetic lipid molecules including glycerophospholipids, glyceroglycolipids, sphingophospholipids and sphingoglycolipids, and from the classes phosphatidyl choline, phosphatidyl ethanolamine, phosphatidyl serine, phosphatidyl glycerol, phosphatidyl acid, phosphatidyl inositol, galactopyranoside, glucopyranoside, digalactopyranoside, diglucopyranoside, ceramide-phosphatidyl choline, ceramide-phosphatidyl ethanolamine, ceramide-phosphatidyl serine, ceramide-phosphatidyl glycerol, ceramide-phosphatidyl acid, ceramide-phosphatidyl inositol, sphingomyelin molecules, glucosylceramides, glucocerebrosides, galactoceramides, galactocerebrosides, gangliosides, monoacyl phosphatidyl choline, cardiolipin molecules, that may be linked to saturated or mono-, di or polyunsaturated fatty or fluorocarbon chains ranging from three to thirty carbons in length where fatty chains attached to the head group can be the same or of different structure, cholesterol, lanosterol, ergosterol, stigmasterol, sitosterol and derivatives thereof capable of being incorporated into lipid membranes, N,N-dimethyl-N-octadecyl-1-octadecanammonium chloride or bromide, (N-[1-(2,3-dioleoyloxy)propyl]-N,N,N-trimethylammonium chloride, N-[2,3-dihexadecyloxy)prop-1-yl]-N,N,N-trimethylammonium chloride, bolaamphiphiles, polyglycerolmonoalkylethers, polyethoxymonoalkylethers, and liposome-forming molecules from the classes amphiphilic polymers, amino acids, crown ether compounds and di(acyloxy)dialkylsilanes; and mixtures thereof.

38. The method according to claim 1, wherein the lipid is selected from phosphatidylcholines with acyl chains ranging in length from 14 to 18 carbons, including di-1,2-myristoyl-SN-phosphatidylcholine, di-1,2-oleoyl-SN-phosphatidylcholine, B-palmitoyl-2-oleoyl-SN-phosphatidylcholine (POPC) and 1-stearoyl-2-oleoyl-SN-phosphatidylcholine; glyceroglycolipids, including di-1,2-myristoyl-3-diglucopyranosyl-SN-glycerol, di-1,2-oleoyl-3-diglucopyranosyl-SN-glycerol, 1-palmitoyl-2-oleoyl-3-diglucopyranosyl-SN-glycerol, 1-stearoyl-2-oleoyl-3-diglucopyranosyl-SN-glycerol, di-1,2-myristoyl-3-digalactopyranosyl-SN-glycerol, di-1,2-oleoyl-3-digalactopyranosyl-SN-glycerol, 1-palmitoyl-2-oleoyl-3-digalactopyranosyl-SN-glycerol and 1-stearoyl-2-oleoyl-3-digalactopyranosyl-SN-glycerol; sphingomyelins with the corresponding acyl chain lengths and unsaturations; and mixtures thereof.

39. The method according to claim 1, wherein the detergent is selected from 3-[(3-cholamidopropyl)dimethylammonio]-1-propane sulfonate (CHAPS), 3-[(3-cholamidopropyl)dimethylammonio]-2-hydroxy-1-propane sulfonate (CHAPSO), N,N-bis-(3-D-gluconeamidopropyl)-deoxycholamide (deoxy-BIGCHAP), sodium taurocholate, cholic acid, deoxycholic acid, n-octylglucoside (OG), n-octylthioglucoside, N-decyl-N,N-dimethyl-3-ammonio-1-propane sulfonate (Zwittergent 3-10), N-dodecyl-N,N-dimethyl-3-ammonio-1-propane sulfonate (Zwittergent 3-12), octanoyl-N-methylglucamide (MEGA-8), decanoyl-N-methylglucamide (MEGA-10), 6-O-(N-heptylcarbamoyl)-methyl-α-D-glucopyranoside (HECAMEG), sucrose monolaurate, and mixtures thereof.

40. The method according to claim 1, wherein the concentration of the lipid in the aqueous liquid containing detergent/lipid mixed micelles is from about 0.1 to about 50 mM.

41. The method according to claim 1, wherein the concentration of the lipid in the aqueous liquid containing detergent/lipid mixed micelles is from about 0.1 to about 10 mM.

42. The method according to claim 1, wherein the concentration of the detergent in the aqueous liquid containing detergent/lipid mixed micelles is from about 0.5×CMC to about 10×CMC for the detergent.

43. The method according to claim 1, wherein the concentration of the detergent in the aqueous liquid containing detergent/lipid mixed micelles is from about 0.5×CMC to about 5×CMC for the detergent.

44. Use of the method according to claim 1 for reconstituting protein function.

45. A substrate surface supporting a lipid film membrane structure as prepared in claim 1.

46. Use of a substrate surface supporting a lipid film membrane structure as prepared in claim 1 for studies of molecular interactions therewith.

47. The use according to claim 46 for studies of interactions with species selected from membrane associated proteins and peptides.

48. The use according to claim 46 for membrane absorption studies.

49. The use according to claim 46 for drug screening.

50. A method of preparing a substrate surface supporting a lipid film membrane structure, which method comprises the steps of:
a) in a flow cell contacting the substrate surface with an aqueous liquid flow containing detergent/lipid mixed micelles to adhere detergent/lipid mixed micelles to the substrate surface, and
b) contacting the substrate surface having detergent/lipid mixed micelles adhered thereto in the flow cell with an aqueous liquid flow substantially free from detergent to elute the detergent molecules from the adhered mixed micelles and make the remaining lipid molecules assemble into a lipid film membrane structure on the substrate surface.

51. A method of preparing a substrate surface supporting a lipid film membrane structure, which method comprises the steps of:
a) providing a substrate surface having a biomolecule immobilized thereon,
b) contacting the substrate surface with an aqueous liquid flow containing detergent/lipid mixed micelles to adhere detergent/lipid mixed micelles to the substrate surface, and
c) contacting the substrate surface having detergent/lipid mixed micelles adhered thereto with an aqueous liquid flow substantially free from detergent to elute the detergent molecules from the adhered mixed micelles and make the remaining lipid molecules assemble into a lipid film membrane structure on the substrate surface.

52. A substrate surface for use in surface reconstitution of a protein or a polypeptide, comprising a hydrogel modified with lipophilic compounds, and immobilized to the hydrogel one member of a specific binding pair, the other member of the specific binding pair being attached to or part of a protein or peptide to be reconstituted on the surface.

53. The substrate surface according to claim 52, wherein the hydrogel comprises carboxymethylated dextran having glucose moieties substituted with lipid groups.

54. The substrate surface according to claim 52, wherein the hydrogel comprises carboxymethylated dextran having glucose moieties substituted with alkyl groups.

55. The substrate surface according to claim 52, wherein the immobilized specific binding pair member is an antibody.

56. The substrate surface according to claim 52, wherein the immobilized specific binding pair member is a monoclonal antibody.

57. The substrate surface according to claim 52, wherein the immobilized specific binding pair member is selected from avidin and streptavidin.

58. The substrate surface according to claim 52, wherein the immobilized specific binding pair member is a metal chelate.
